(19) United States
(12) Patent Application Publication
(10) Pub. No.: US 2012/0059360 A1
(43) Pub. Date: Mar. 8, 2012

(54) SURGICAL POWER TRANSMISSION ADAPTER AND MEDICAL MANIPULATOR SYSTEM

(75) Inventor: Hirotaka NAMIKI, Kodaira-shi (JP)

(73) Assignee: OLYMPUS CORPORATION, Tokyo (JP)

(21) Appl. No.: 13/074,160

(22) Filed: Mar. 29, 2011

(30) Foreign Application Priority Data
Sep. 6, 2010 (JP) 2010-199231

(51) Int. Cl. A61B 17/00

(52) U.S. Cl. 606/1

(57) ABSTRACT

A surgical power transmission adapter is interposed between a surgical instrument and a power unit configured to drive the surgical instrument. The adapter connects the surgical instrument to the power unit. The adapter includes a power transmission unit. The power transmission unit includes a first region which is in contact with a clean region that is subjected to sterilization processing and a second region which is in contact with an unclean region that is not subjected to the sterilization processing. The unit makes translatory movement to transmit power generated in the power unit to the surgical instrument. A range of the translatory movement of the power transmission unit is set in such a manner that the first region is placed in the clean region and the second region is placed in the unclean region even if the power transmission unit makes the translatory movement.

### CROSS-REFERENCE TO RELATED APPLICATIONS

This application is based upon and claims the benefit of priority from prior Japanese Patent Application No. 2010-199231, filed Sep. 6, 2010, the entire contents of which are incorporated herein by reference.

### BACKGROUND OF THE INVENTION

1. Field of the Invention

The invention relates to a surgical power transmission adapter for use in a medical manipulator system and the medical manipulator system including the surgical power transmission adapter.

2. Description of the Related Art

In recent years, to reduce personnel in medical facilities, medical treatments given by robots have been studied. Particularly, in the surgical field, various suggestions about a medical manipulator system have been made. The medical manipulator system is configured to give a treatment for a patient based on a multi-degree-of-freedom manipulator including a multi-degree-of-freedom arm. Among others, in Internal Publication No. 2006-053198, a connector that transmits power for driving an arm or a surgical instrument attached to an end of the arm by translatory movement (a surgical power transmission adapter) has been suggested.

There are various kinds of surgical instruments attached to the end of the arm. Of these instruments, a surgical instrument such as a scalpel or surgical scissors directly comes into contact with a body cavity of a patient. Therefore, such a surgical instrument must be sterilized before use. As sterilization process systems, there are various kinds of systems such as autoclave sterilization or EOG sterilization. According to the autoclave sterilization, bacteria are destroyed by saturated water vapor having high temperature and high pressure. According to the EOG sterilization, bacteria are destroyed by alkylation using an ethylene oxide gas (EOG). Usually, a power unit configured to drive an arm and others in the medical manipulator system does not have a structure that can bear with such various sterilization process systems. Therefore, a sterilization process is carried out with a unit having no structure that can bear with the sterilization process, e.g., the power unit being separated from a part that must be sterilized.

### BRIEF SUMMARY OF THE INVENTION

According to a first aspect of the invention, there is provided a surgical power transmission adapter that is interposed between a surgical instrument and a power unit configured to drive the surgical instrument and connects the surgical instrument to the power unit, comprising: a power transmission unit that includes a first region which is in contact with a clean region that is subjected to sterilization processing and a second region which is in contact with an unclean region that is not subjected to the sterilization processing, and that is configured to make translatory movement to transmit power generated in the power unit to the surgical instrument, wherein a range of the translatory movement of the power transmission unit is set in such a manner that the first region is placed in the clean region and the second region is placed in the unclean region even if the power transmission unit makes the translatory movement.

According to a second aspect of the invention, there is provided a medical manipulator system comprising: a surgical power transmission adapter comprising: a power transmission unit that includes a first region which is in contact with a clean region that is subjected to sterilization processing and a second region which is in contact with an unclean region that is not subjected to the sterilization processing, and that is configured to make translatory movement, a range of the translatory movement being set in such a manner that the first region is placed in the clean region and the second region is placed in the unclean region even if the power transmission unit makes the translatory movement; a power unit that is connected to the surgical power transmission adapter and configured to transmit generated power by the translatory movement of the power transmission unit; a surgical instrument that is connected to the surgical power transmission adapter and configured to be driven by the power transmitted based on the translatory movement of the power transmission unit; and a control circuit that is connected to the power unit and configured to control operations of the surgical instrument by controlling the power unit.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing a first example of a medical manipulator system according to each embodiment of the invention;

FIG. 2 is a view showing a second example of the medical manipulator system according to each embodiment of the invention;

FIG. 3 is a view showing a configuration of a surgical power transmission adapter according to a first embodiment of the invention;

FIG. 4A and FIG. 4B are views showing a configuration of a surgical power transmission adapter according to a modification of the first embodiment of the invention;

FIG. 5A and FIG. 5B are views showing a modification of a coupling structure between rods;

FIG. 6 is a view showing a configuration of a surgical power transmission adapter according to a second embodiment of the invention;

FIG. 7A and FIG. 7B are views showing key pattern units;

FIG. 8 is a view showing a configuration of a surgical power transmission adapter according to a modification of the second embodiment of the invention;

FIG. 9 is a first view showing a modification that a memory is mounted in a surgical instrument; and

FIG. 10 is second view showing a modification that a memory is mounted in a surgical instrument.

### DETAILED DESCRIPTION OF THE INVENTION

Embodiments according to the invention will now be described hereinafter in detail.

### First Embodiment

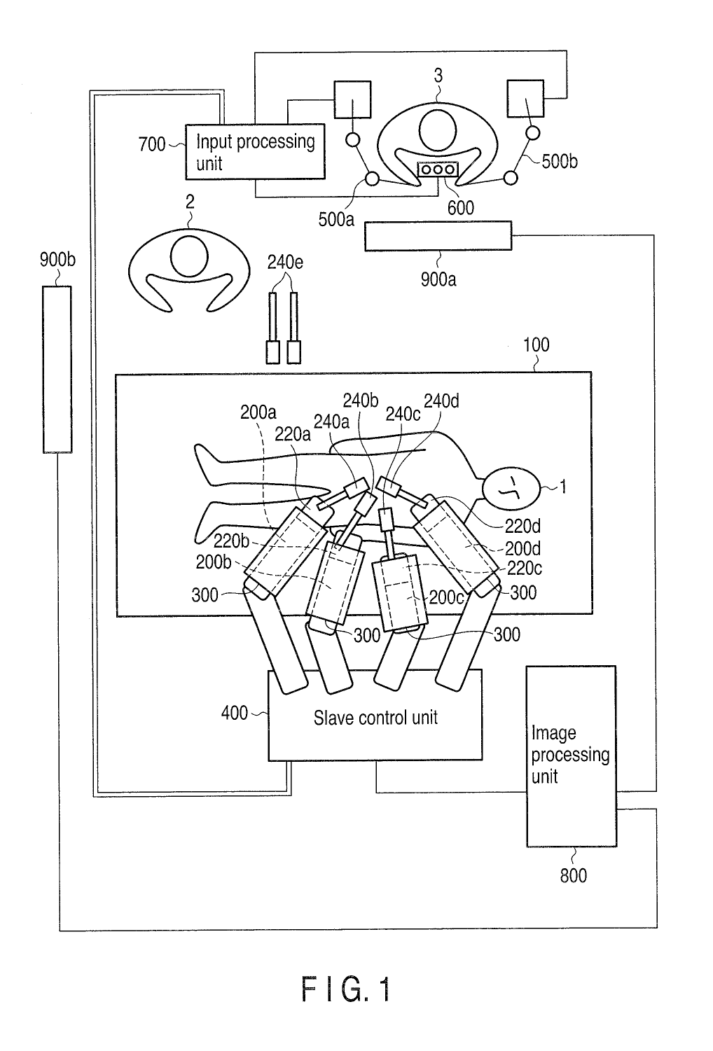

A first embodiment of the invention will now be described. FIG. 1 is a view showing a modification of a medical manipulator system according to each embodiment of the invention. FIG. 1 shows an example of the application to a medical manipulator system adopting a master-slave system. Here, the medical manipulator system adopting the master-slave system means a system that includes a two types of arms including a master arm and a slave arm and remotely controls the slave arm to follow an operation of the master arm.

The medical manipulator system depicted in FIG. 1 has an operating table 100, slave arms 200a to 200d, a slave control unit 400, master arms 500a and 500b, an operation unit 600, an input processing unit 700, an image processing unit 800, and displays 900a and 900b.

The operating table 100 is a table on which a patient 1 who is an observation/treatment target lies. The plurality of slave arms 200a, 200b, 200c, and 200d are disposed near the operating table 100. Slave arms 200a to 200d may be disposed on the operating table 100.

Each of slave arms 200a, 200b, 200c, and 200d is configured to have a plurality of multi-degree-of-freedom joints. Each of slave arms 200a, 200b, 200c, and 200d bends each joint to position each of various surgical instruments such as a treatment instrument or an observation instrument attached on an end side of each of slave arms 200a to 200d with respect to the patient 1 who lies on the operating table 100. The end side is determined as a side facing a body cavity of the patient 1. Each joint of slave arms 200a to 200d is individually driven by a power unit provided in each arm. As the power unit, for example, a motor (a servo motor) having a servo mechanism including an incremental encoder or a decelerator is used. An operation of the servo motor is controlled by the slave control unit 400.

Furthermore, slave arms 200a to 200d also include a plurality of power units configured to drive surgical instruments 240a to 240d attached on the respective end sides. As this power unit, for example, a servo motor is likewise used. An operation of this servo motor is also controlled by the slave control unit 400.

When the power units of slave arms 200a to 200d are driven, driving amounts of the motors are detected by a position detector. A detection signal from the position detector is input to the slave control unit 400. Based on this detection signal, the driving amounts of slave arms 200a to 200d are detected by the slave control unit 400.

Surgical power transmission adapters (which will be simply referred to as an adapters hereinafter) 220a, 220b, 220c, and 220d are interposed between slave arms 200a, 200b, 200c, and 200d and surgical instruments 240a, 240b, 240c, and 240d, respectively. As a result, adapters 220a, 220b, 220c, and 220d connect slave arms 200a, 200b, 200c, and 200d with surgical instruments 240a, 240b, 240c, and 240d. As will be described later in detail, each of adapters 220a to 220d has a translatory mechanism and is configured to transmit power generated in the power unit in the corresponding slave arm to the corresponding surgical instrument by translatory movement.

Each of surgical instruments 240a to 240d has joint portions having a plurality of freedom degrees and is inserted into a body cavity of the patient 1 from a non-illustrated insertion hole formed in a body wall of the patient 1. Moreover, an end portion of each of surgical instruments 240a to 240d is configured to be bent and/or rotated. This bending driving is carried out by, e.g., driving the servo motor provided in each of slave arms 200a to 200d to push or pull a wire or a rod inserted and arranged in each of surgical instruments 240a to 240d. Additionally, the rotational driving is carried out by, e.g., driving the servomotor provided in each of slave arms 200a to 200d to operate a rotating mechanism provided in each of surgical instruments 240a to 240d. Further, an opening/closing mechanism may be provided at an end of each surgical instrument depending on the type of surgical instrument. This opening/closing mechanism is operated by, e.g., driving the servo motor provided in each of slave arms 200a to 200d to push or pull the wire or the rod inserted and arranged in the surgical instrument.

Of the four slave arms 200a to 200d depicted in FIG. 1, for example, slave arms 200a, 200b, and 200d are used as slave arms for treatments. Various kinds of surgical instruments are attached as surgical instruments 240a, 240b, and 240d to these slave arms 200a, 200b, and 200d for treatments. The surgical instrument in this embodiment means a surgical instrument used for giving a treatment or performing an operation with respect to a tissue site in a body of the patient 1. Furthermore, slave arm 200c is used as a camera arm for observation. Various kinds of observation instruments are attached as surgical instrument 240c to slave arm 200c. The observation instrument in this embodiment means a surgical instrument for observing a tissue site in a body of the patient 1.

Surgical instruments 240a to 240d attached to adapters 220a to 220d can be replaced with replacement surgical instruments 240e. A replacement operation of such surgical instruments is carried out by, e.g., an assistant 2.

Each drape 300 is used for discriminating a region that is to be sterilized (which will be referred to as a clean region hereinafter) and a region that is not to be sterilized (which will be referred to as an unclean region hereinafter) from each other in this medical manipulator system. For example, a surgical instrument such as a scalpel or surgical scissors directly comes into contact with a body cavity of the patient 1, and it must be sufficiently cleaned or sterilized. On the other hand, the power units and others of slave arms 200a to 200d include various kinds of electronic components, and hence they are not configured to resist the sterilization processing. Therefore, when performing an operation with surgical instruments 240a to 240d being attached to slave arms 200a to 200d through adapters 220a to 220d, sterilized parts of surgical instruments 240a to 240d are exposed to protect the power units of slave arms 200a to 200d. Therefore, as shown in FIG. 1, the operation is performed in a state that the drapes 300 are wrapped around the power units of slave arms 200a to 200d. It is to be noted, when each drape 300 is utilized to discriminate the clean region and the unclean region from each other, mixture of the clean region and the unclean region is avoided after the sterilization processing. A necessary minimum range may be draped as shown in FIG. 1, or a wider range reaching, e.g., the slave control unit 400 may be covered with the drape 300.

The slave control unit 400 is configured with, e.g., a CPU or a memory. This slave control unit 400 stores a predetermined program utilized to control slave arms 200a to 200d. The slave control unit 400 controls operations of slave arms 200a to 200d or surgical instruments 240a to 240d in accordance with control signals from the input processing unit 700. The slave control unit 400 specifies the slave arm (or the surgical instrument) as an operation target of the master arm operated by an operator 3. Furthermore, the slave control unit 400 calculates a driving amount required for movement associated with an operation amount of the master arm of the operator 3. Moreover, the slave control unit 400 controls an operation of the slave arm as the operation target of the master arm in accordance with the calculated driving amount. At this time, the slave control unit 400 inputs a driving signal to the corresponding slave arm. Additionally, the slave control unit 400 controls an intensity or a polarity of the driving signal in such a manner that the driving amount for the slave arm as the operation target becomes a target driving amount in accordance with a detection signal input from the positional detector of the power unit based on an operation of the corresponding slave arm.

Further, when an image signal is input from the observation instrument attached to slave arm 200c, the slave control unit 400 outputs the input image signal to the image processing unit 800. The observation instrument may be directly connected to the image processing unit without interposing the slave control unit 400.

Each of master arms 500a and 500b includes a plurality of link mechanisms. A positional detector such as an incremental encoder is provided to each link including the link mechanism. When this positional detector detects an operation of each link, the input processing unit 700 detects an operation amount of each of master arms 500a and 500b.

In the example of FIG. 1, master arm 500a is an arm operated by a right hand of the operator 3, and master arm 500b is an arm operated by a left hand of the operator 3. FIG. 1 shows an example when the two master arms 500a and 500b are utilized to operate the four slave arms. In this case, the slave arm as the operation target of the master arm needs to be appropriately switched. Such switching is performed by, e.g., an operation of the operation unit 600 of the operator 3. Of course, when the operation target has a one-to-one relationship by setting the number of the master arms to be equal to the number of the slave arms, such switching is not required.

The operation unit 600 includes various kinds of operation members such as a switching button for switching the slave arm as an operation target of master arm 500a or 500b (which will be referred to as a switching button), a scaling change switch used for changing an operation ratio of the master and the slave, a foot switch for emergency stop of the system, and others. When the operator 3 has operated any operation member including the operation unit 600, an operation signal associated with an operation of the corresponding operation member is input from the operation unit 600 to the input processing unit 700.

The input processing unit 700 analyzes operation signals from master arms 500a and 500b and an operation signal from the operation unit 600. Furthermore, the input processing unit 700 generates a control signal that is utilized to control the medical manipulator system in accordance with an analysis result of the operation signals and inputs the generated control signal to the slave control unit 400.

The image processing unit 800 performs various kinds of image processing required to display an image signal input from the slave control unit 400. As a result, image data for display in an operator display 900a and an assistant display 900b are generated. Each of the operator display 900a and the assistant display 900b is constituted of, e.g., a liquid crystal display. Each of these displays 900a and 900b displays an image based on image data generated by the image processing unit 800 in accordance with the image signal acquired through the observation instrument. The image can be simply displayed if it is a two-dimensional image, or depth feel can be obtained if the image is a three-dimensional image.

In the medical manipulator system having the configuration depicted in FIG. 1, surgical instruments 240a to 240d are sterilized. As the sterilization processing in this embodiment, various kinds of systems such as autoclave sterilization or EOG sterilization can be used.

After the sterilization processing, the operator 3 operates master arms 500a and 500b while watching an image displayed in the operator display 900a. This image is displayed based on the image signal fetched through the observation instrument attached to the end of slave arm 200c. In response to operations of master arms 500a and 500b performed by the operator 3, a detection signal from the positional detector disposed to each link of master arms 500a and 500b is input to the input processing unit 700. Further, when the switching button of the operation unit 600 is operated, an operation signal from the operation unit 600 is input to the input processing unit 700.

The input processing unit 700 counts the number of times of input of the operation signals from the switching button of the operation unit 600. The input processing unit 700 switches the slave arm as an operation target of each of master arms 500a and 500b in accordance with the number of times of input of the operation signals. For example, in an initial state, the operation target of master arm 500a is determined as slave arm 200a, and the operation target of master arm 500b is determined as slave arm 200b. In the initial state, when the switching button is pressed once, the input processing unit 700 switches the operation target of master arm 500a to slave arm 200c or switches the operation target of master arm 500b to slave arm 200d. Thereafter, every time the switching button is pressed, the input processing unit 700 switches the operation target of master arm 500a between slave arm 200a and slave arm 200c or switches the operation target of master arm 500b between slave arm 200b and slave arm 200d.

Additionally, when a detection signal is input from the positional detector in one of master arms 500a and 500b, the input processing unit 700 determines an operation amount of the master arm from a value of the detection signal. Moreover, the input processing unit 700 generates a control signal including information indicative of the operation amount of the master arm operated by the operator 3 and information required to determine the slave arm as the operation target of the operated master arm, and the generated control signal is input to the slave control unit 400.

The slave control unit 400 calculates a driving amount required for the slave manipulator to effect movement associated with the operation of the master arm performed by the operator 3 in accordance with the control signal from the input processing unit 700. Additionally, the slave control unit 400 controls an intensity or a polarity of a driving signal that is input to the slave arm as the operation target in such a manner that a driving amount of the slave arm as the operation target of the master arm reaches the calculated driving amount.

Further, the slave control unit 400 stops slave arms 200a to 200d when the operator 3 presses the foot switch and a control signal indicative of emergency stop of the system is input from the input processing unit 700.

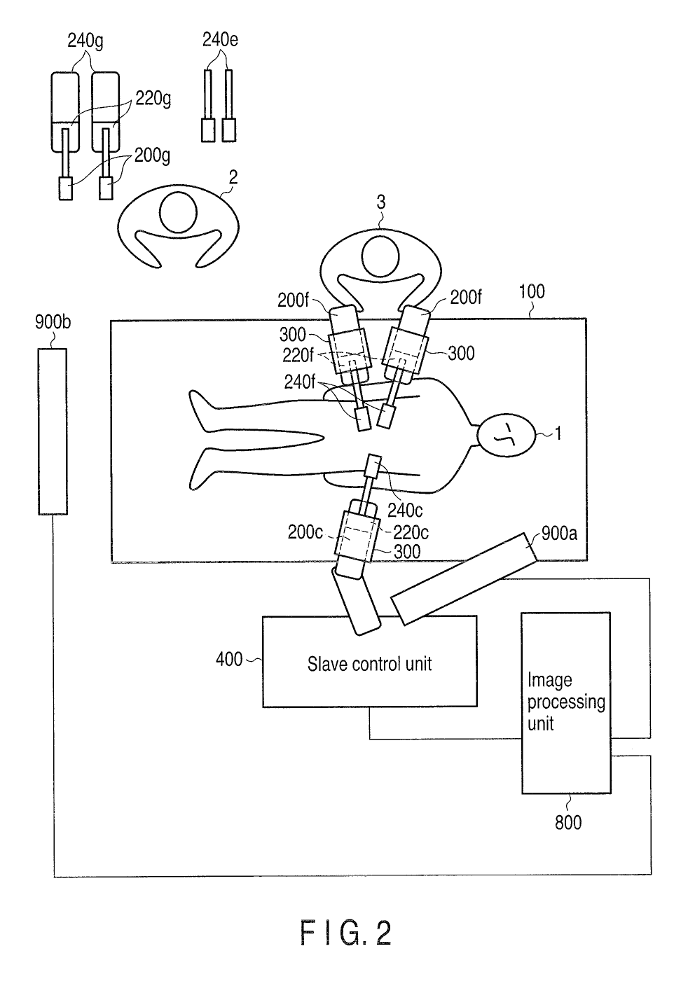

FIG. 2 is a view showing a second example of the medical manipulator system according to each embodiment of the invention. FIG. 2 shows an example of the application to a handy-type medical manipulator system. Here, the handy-type medical manipulator system means a system that each arm including a surgical instrument attached thereto is directly operated by the operator 3.

In FIG. 2, reference numerals equal to those in FIG. 1 denote structures equal to or corresponding to those in FIG. 1, thereby omitting a description thereof. A slave control unit 400 depicted in FIG. 2 is provided to control an operation of an arm 200c as a camera arm in a master-slave system. If the arm 200c is also a handy-type arm, the slave control unit 400 is not required.

Handy arms 200f are disposed near the operating table 100 like slave arms 200a to 200d. Each handy arm 200f includes a plurality of power units configured to drive a surgical instrument connected to an end side like slave arms 200a to 200d. The power units in the handy arm 200f generate power in accordance with a driving signal from a non-illustrated operation unit provided to the handy arm 200f. Further, as shown in FIG. 2, the same adapter 220f as that depicted in FIG. 1 is connected to the end of the handy arm 200f, and a surgical instrument 240f is connected to adapter 220f.

The handy arm 200f, adapter 220f, and surgical instrument 240f can be replaced with other handy arm 200g, adapter 220g, and surgical instrument 240g, respectively. A replacement operation for such surgical instrument is carried out by, e.g., the assistant 2.

In the medical manipulator system having the configuration depicted in FIG. 2, the operator 3 operates the non-illustrated operation unit of the handy arm 200f, thereby driving the handy arm 200f or surgical instrument 240f. As other operations, the operations explained in FIG. 1 are applied.

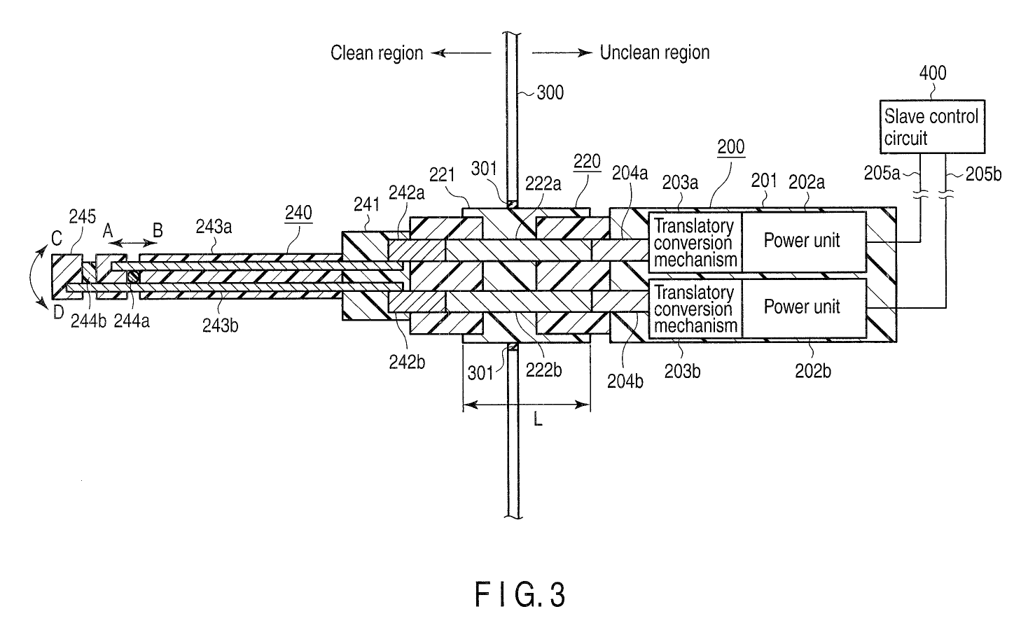

The surgical power adapter according to this embodiment will be further described hereinafter. FIG. 3 is a view showing a configuration of the surgical power transmission adapter according to a first embodiment of the invention. It is to be noted that the adapter in FIG. 3 can be applied to both the medical manipulator systems having the configurations shown in FIG. 1 and FIG. 2. Furthermore, FIG. 3 shows the configuration around the adapter alone and, for example, joints included in the arm 200 itself are omitted in the drawing.

The arm 200 shown in FIG. 3 includes an arm main body 201, power units 202a and 202b, translatory conversion mechanisms 203a and 203b, and rods 204a and 204b.

The arm main body 201 is made of, e.g., a metal material and/or a resin material. In this arm main body 201, spaces accommodating power units 202a and 202b, translatory conversion mechanisms 203a and 203b, and rods 204a and 204b are formed.

Each of power units 202a and 202b includes a servo motor having a servo mechanism such as a positional detector and a decelerator. Each of power units 202a and 202b is electrically connected to the slave control unit 400 through wiring lines 205a and 205b. Operations of power units 202a and 202b are controlled by the slave control unit 400. In FIG. 3, each of wiring lines 205a and 205b is shown as one wiring line. Actually, each of wiring lines 205a and 205b includes a plurality of wiring lines, e.g., a wiring line used for transmitting a driving signal, a wiring line used for transmitting a detection signal from the positional detector, a wiring line for earth, and others. The arm 200 shown in FIG. 3 is configured to drive the joins of the surgical instrument 240 by pushing or pulling the rods arranged to be inserted in the arm main body 201.

Each of translatory conversion mechanisms 203a and 203b includes a ball screw and others. Translatory conversion mechanisms 203a and 203b convert rotational power of the motors in power units 202a and 202b into translatory movement. Rods 204a and 204b as translatory mechanisms are coupled with translatory conversion mechanisms 203a and 203b. Rods 204a and 204b transmit the translatory power converted by the respective translatory conversion mechanisms 203a and 203b to the adapter 220.

The adapter 220 includes an adapter main body 221 and rods 222a and 222b.

The adapter main body 221 is made of a material that can resist the sterilization processing, e.g., a metal material or a resin material. For example, in the case of autoclave sterilization, a material that can resist high temperature and high pressure is used. This adapter main body 221 has a hollow structure that accommodates rods 204a and 204b as the power transmitting units. Moreover, the adapter main body 221 is configured to be detachable with respect to the arm main body 201 and also configured to be detachable with respect to a surgical instrument main body 241. When the adapter main body 221 is attached to the arm main body 201 or the surgical instrument main body 241, for example, a non-illustrated locking mechanism provided to the adapter main body 221 holds the adapter main body 221. Moreover, in this embodiment, as shown in FIG. 3, the drape 300 is configured to hold the adapter main body 221 in an opening portion formed in the drape 300. For example, as shown in FIG. 3, a rubber ring 301 is formed at the opening portion of the drape 300, and the adapter main body 221 is held by elasticity of this rubber ring 301. A groove and the like may be formed in the adapter main body 221 so that the adapter main body 221 can be more assuredly held in the opening portion of the drape 300.

When the sterilization processing is performed in such a configuration, the adapter main body 221 substantially entirely has the clean region after the sterilization processing, but a part of the adapter main body 221 that has come into contact with an unclean region during use or the like becomes an unclean region. In this manner, the adapter main body 221 is divided into the clean region and the unclean region with the drape 300 at a boundary.

Rods 222a and 222b as the power transmitting portions are configured to be coupled with rods 204a and 204b in the arm main body 201, respectively, when the adapter main body 221 is attached to the arm main body 201. Rods 222a and 222b make translatory movements with translatory movements of rods 204a and 204b. Additionally, rods 222a and 222b transmit power generated in power units 202a and 203b to the surgical instrument 240.

The surgical instrument 240 includes the surgical instrument main body 241, rods 242a and 242b, wires 243a and 243b, joints 244a and 244b, and an end portion 245. Joints 244a and 244b are orthogonal joints.

The surgical instrument main body 241 is made of a material that can resist the sterilization processing, e.g., a metal material or a resin material or a combination thereof. Further, the surgical instrument main body 241 contains rods 242a and 242b and wires 243a and 243b.

Rods 242a and 242b are configured to be coupled with rods 222a and 222b in the adapter main body 221 when the surgical instrument main body 241 is attached to the adapter main body 221. Rods 242a and 242b make the translatory movements with the translatory movements of rods 222a and 222b. Wires 243a and 243b are coupled with rods 242a and 242b. Wires 243a and 243b push or pull the end portion 245 with the translatory movements of rods 222a and 222b. Joints 244a and 244b are interposed between the surgical instrument main body 241 and the end portion 245. Joints 244a and 244b rotate with the operations of wires 243a and 243b. The end portion 245 including various kinds of surgical instruments or observation instruments attached thereto is driven to be bent while being guided by rotation of joints 244a and 244b.

In the configuration shown in FIG. 3, for example, when rod 204a is driven in the direction of arrow B in the drawing and rod 204b is driven in the direction of arrow A in the drawing, rods 222a and 242a make the translatory movements in the direction B with the translatory movement of rod 204a in the direction of arrow B in the drawing. Likewise, rods 222b and 242b also make the translatory movements in the direction A with the translatory movement of rod 204b in the direction of arrow A in the drawing. As a result, wire 243a coupled with rod 242a is driven to pull the end portion 245. On the other hand, wire 243b coupled with rod 242b is driven to push out the end portion 245. Therefore, the end portion 245 is driven in the direction of arrow C in the drawing and a paper sheet surface direction (not shown). Contrarily, when rod 204a is driven in the direction of arrow A in the drawing and rod 204b is driven in the direction of arrow B in the drawing, the end portion 245 is driven in the direction of arrow D in the drawing and a paper sheet back direction (not shown). In this manner, the end portion 245 can be driven along a pitch direction and a yaw direction.

FIG. 3 shows an example that the end portion 245 is driven in the pitch direction and the yaw direction. However, the driving directions of the end portion 245 are not restricted to these directions, and increasing the numbers of the power units, the translatory conversion mechanisms, and the rods enables raising the number of the driving directions. Furthermore, when a rotation mechanism and the like are provided, the end portion 245 can be rotated.

As described above, rod 222a of the adapter 220 is coupled with both rod 204a of the arm 200 and rod 242a of the surgical instrument 240. Moreover, these rods can be integrally driven. Likewise, rod 222b of the adapter 220 is coupled with both rod 204b of the arm 200 and rod 242b of the surgical instrument 940. Additionally, these rods are integrally driven.

In this embodiment, the range of the translatory movement of rod 222a is set in such a manner that a first region in rod 222a which is a coupling portion with respect to rod 242a does not come into contact with a wall surface (i.e., the unclean region) of the adapter main body 221 on the arm 200 side even when rod 222a, rod 204a, and rod 242a make the maximum translatory movements in a pulling direction (the direction of arrow B in the drawing) and in such a manner that a second region in rod 222a which is a coupling portion with respect to rod 204a does not come into contact with a wall surface (i.e., the clean region) of the adapter main body 221 on the surgical instrument 240 side even when rod 222a, rod 204a, and rod 242a make the maximum translatory movements in a pushing direction (the direction of arrow A in the drawing). The range of the translatory movement of rod 222b is similarly set. Furthermore, the movable ranges of the first region and the second region are prevented from being mixed.

Actually, the requested movable range of the end portion 245 differs depending on the type of surgical instrument 240. Therefore, the driving ranges of rods 222a and 222b need to differ depending on the movable range of the end portion 245. Therefore, it is desirable to use the adapter 220 that the driving ranges of rods 222a and 222b are appropriately set in accordance with the type of surgical instrument 240. For example, when increasing the movable range of the end portion 245, the driving ranges of rods 222a and 222b need to be thereby enlarged. Therefore, the driving ranges of rods 222a and 222b are set to meet conditions for the movable range of the end portion 245. Moreover, a dimension L in the adapter main body 221 along the direction of translatory movement of the rods is set in such a manner that the first region does not come into contact with the unclean region of the adapter main body 221 and the second region does not come into contact with the clean region of the adapter main body 221.

As described above, in this embodiment, the ranges of the translatory movements of rod 221a and rod 222b are set in such a manner that rods 242a and 242b of the surgical instrument 240 do not come into contact with the wall surface (the unclean region) of the adapter main body 221 on the arm 200 side and rods 204a and 204b of the arm 200 do not come into contact with the wall surface (the clean region) of the adapter main body 221 on the surgical instrument side, at the time of driving the arm 200. Moreover, the movable ranges of the first region and the second region are prevented from being mixed. Therefore, in the medical manipulator that transmits the power by the translatory movement, the clean region and the unclean region is not mixed at the time of driving the arm 200. As a result, the surgical instrument 240 can be held in the clean state. Additionally, when the adapter 220 is interposed between the arm 200 and the surgical instrument 240, the surgical instrument 240 can be combined with the arm 200 having a structure that cannot resist the sterilization processing to be used for a surgical operation by sterilizing the surgical instrument 240 and the arm 200. Further, the adapter 220 transmits the power generated by the power units in the form of the translatory movement. Therefore, a diameter can be relatively easily reduced.

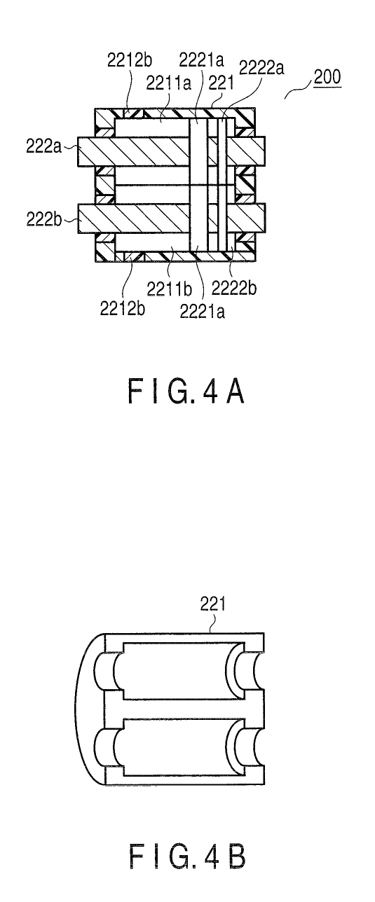

A modification of the first embodiment will now be described hereinafter. FIG. 4A is a view showing a configuration of an adapter 220 according to a first modification of the first embodiment. As shown in FIG. 4A, rods 222a and 221b are accommodated in an adapter main body 221 of the adapter 220 according to the first modification. Furthermore, hollow portions 2211a and 2211b that also function as sterilization spaces for the sterilization processing are formed in the adapter main body 221. Moreover, ventilation holes 2212a and 2212b through which a sterilization gas (high-temperature vapor in the case of autoclave sterilization, or an EOG in the case of gas sterilization) flows into hollow portions 2211a and 2211b are formed in the adapter main body 221. When ventilation holes 2212a and 2212b are formed, not only a surface of the adapter main body 221 but also a substantially entire region in the adapter main body 221 can be sterilized in the sterilization processing.

As shown in FIG. 4A, partition walls 2221a and 2222a are integrally formed to rod 222a. Partition walls 2221a and 2222a regulate the driving range of rod 222a and also function as discriminating portions that discriminate a clean region and an unclean region in hollow portion 2211a. Likewise, partition walls 2221a and 2222b are integrally formed to rod 222b. Partition wall portions 2221b and 2222b regulate the driving range of rod 222b and also function as discriminating portions that discriminate a clean region and an unclean region in hollow portion 2211b.

Partition walls 2221a and 2222a are formed into shapes that shield hollow portion 2211a without a gap. Additionally, partition wall 2211a and partition wall 2222a are formed to rod 222a to have a predetermine gap. Based on such a configuration, a surface of partition wall 2221a on the surgical instrument 240 side does not come into contact with partition wall 2222a, and a surface of partition wall 2222a on the arm 200 side does not come into contact with partition wall 2221a. Partition walls 2221b and 2222b are also formed like partition walls 2221a and 2222a.

In such a configuration as depicted in FIG. 4A, at the time of the sterilization processing, rods 222a and 221b are moved to the arm 200 side. As a result, volumes of hollow portions 2211a and 2211b can be increased to sterilize the wide range in the adapter main body 221.

At the time of driving the arm 200, translatory movements of rods 222a and 222b in a pushing direction are regulated by partition wall 2221a and partition wall 2221b, and translatory movements of rods 222a and 222b in a pulling direction are regulated by partition wall 2222a and partition wall 2222b. Therefore, the clean region and the unclean region are not mixed by the translatory movements of rods 222a and 222b.

FIG. 4B is a view showing a configuration of an adapter 220 according to a second modification of the first embodiment. In FIG. 4A, the sterilization processing is carried out in a state that rods 222a and 222b are accommodated in the adapter main body 221. Therefore, the entire ranges of hollow portions 2211a and 2211b in the adapter main body 221 cannot be sterilized. On the other hand, as shown in FIG. 4B, when the adapter main body 221 is configured in a dividable manner, rods 222a and 222b can be removed and then sterilized. In this case, the entire ranges of hollow portions 2211a and 2211b in the adapter main body 221 can be sterilized.

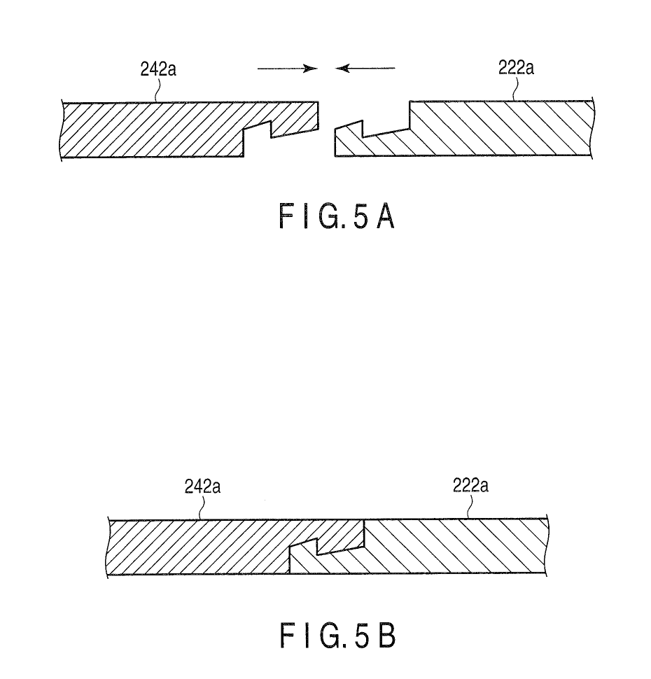

FIG. 5A and FIG. 5B are views showing a modification of a coupling structure between rods. It is to be noted that FIG. 5A and FIG. 5B show a configuration of the coupling portion of a rod 242a and a rod 222a. A coupling portion of a rod 242b and a rod 222b may have the same configuration. A coupling portion of a rod 222a and a rod 204a and a coupling portion of a rod 222b and a rod 204b may also have the same configuration.

As described above, each rod of the adapter 220 in this embodiment is coupled with both the rod of the arm 200 and the rod of the surgical instrument 240. Based on such a configuration, the rod of the adapter 220, the rod of the arm 200, and the rod of the surgical instrument integrally make translatory movements. Various kinds of configurations for coupling these rods can be considered.

For example, as shown in FIG. 5A, an end portion of rod 242a is formed into a hook-like shape, and an end portion of rod 222a is formed into a hook-like shape that engages with the end portion of rod 242a. Providing such a configuration can achieve engagement of the end portion of rod 242a and the end portion of rod 222a as shown in FIG. 5B when the surgical instrument 240 is attached to the adapter 220, thereby coupling rod 242a with rod 222a.

Resides, for example, coupling between the rod of the adapter 220 and the rod of the arm 200 and coupling between the rod of the adapter 220 and the rod of the surgical instrument may be achieved by using magnets. In this case, a magnet can be disposed to an end of the rod of the adapter 220, and the rod of the arm 200 and the surgical instrument 240 can be formed of a ferromagnetic metal such as iron. Besides, the coupling may be achieved by an electromagnetic technique such as electromagnets. Furthermore, an adhesive may be applied to the rod of the adapter 220 and the rod of the arm 200 so that both the rods can be coupled through the adhesive.

### Second Embodiment

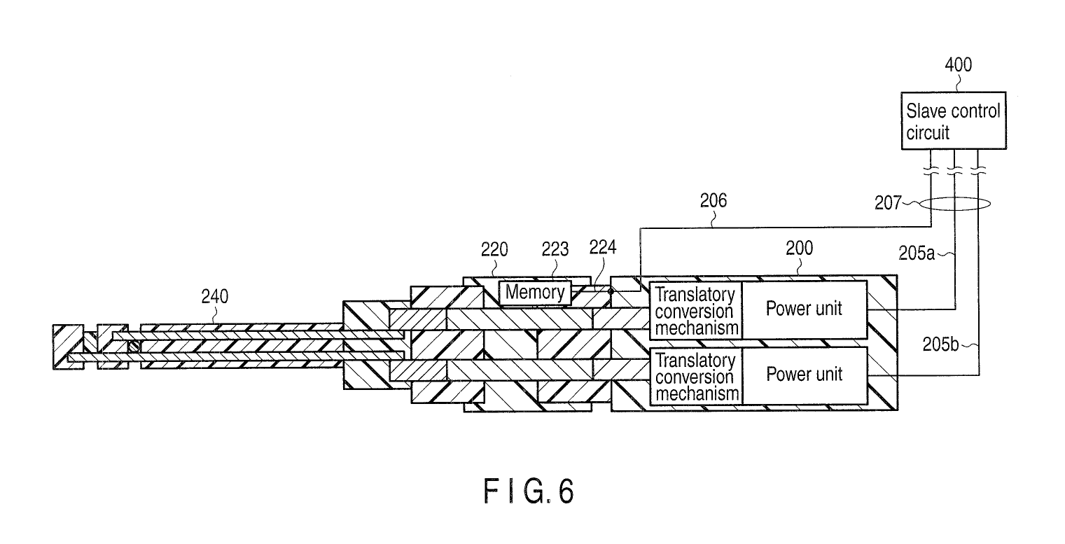

A second embodiment according to the invention will now be described. A surgical power transmission adapter according to the second embodiment can store information required to specify a type of a surgical instrument in addition to having the configuration described in the first embodiment. FIG. 6 is a view showing a configuration of the surgical power transmission adapter according to the second embodiment of the invention. As shown in FIG. 6, an adapter 220 according to this embodiment includes a memory 223. A wiring line 224 is connected to memory 223. Wiring line 224 of memory 223 is configured to be connected to a wiring line 206 provided to an arm 200 when the adapter 220 is connected to the arm 200. Wiring line 206 is let out from an arm main body 201 and connected to a slave control unit 400 together with wiring lines 205a and 205b that are required to drive power units 202a and 202b. As other structures, structures equal to those described in the first embodiment can be applied.

Memory 223 as a surgical instrument identification unit is a memory such as an EEPROM from/into which information can be electrically read/written. This memory 223 stores control parameters in accordance with each type of the surgical instrument 240. The control parameters include, e.g., information indicative of types of the surgical instruments 240.

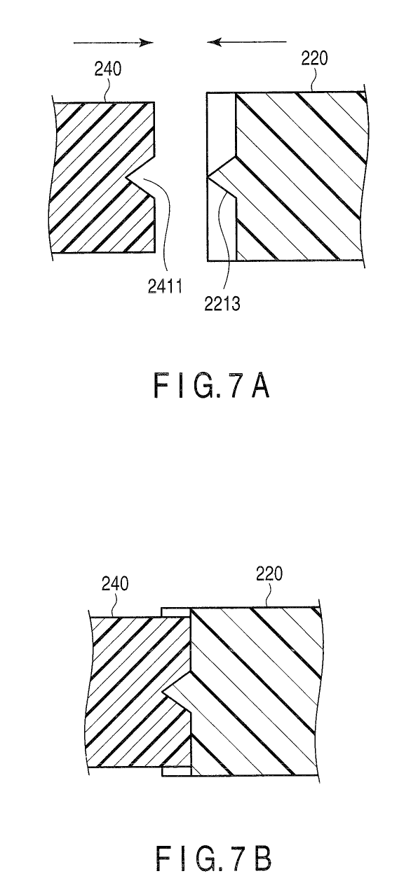

Furthermore, as shown in FIG. 7A, a key pattern portion 2213 having a predetermined shape is formed in an adapter main body 221 according to this embodiment. Moreover, as shown in FIG. 7A, a key pattern portion 2411 having a predetermined shape is formed in a surgical instrument main body 241 according to this embodiment. Here, key pattern portion 2411 is formed to have the same shape in regard to the surgical instruments 240 which are of the same type. Key pattern portion 2213 and key pattern portion 2411 also constitute a surgical instrument identification unit.

Based on such a configuration, a surgical instrument main body 241 including key pattern portion 2411 associated with key pattern portion 2213 alone can be attached to the adapter main body 221. For example, FIG. 7A shows an example that a protruding portion as key pattern portion 2213 is formed in the adapter main body 221 and a groove portion in which the protruding portion formed in the adapter main body 221 is fitted in is formed in the surgical instrument main body 241 as key pattern portion 2411. In this case, as shown in FIG. 7B, key pattern portion 2213 and key pattern portion 2411 are fitted to each other, whereby the surgical instrument main body 211 is attached to the adapter main body 221.

A slave control unit 400 in this embodiment reads the control parameters stored in memory 223 to specify the type of surgical instrument 240. Moreover, the slave control unit 400 controls operations of the surgical instrument 240 in accordance with a control program associated with the specified surgical instrument 240.

As described above, according to the second embodiment, since memory 223 is provided to the adapter 220, the type of surgical instrument 240 can be specified by the slave control unit 400, and the specified surgical instrument 240 can be optimally controlled. As described in the first embodiment, the adapter 220 has a relatively simple configuration that rods 222a and 222b are just accommodated. Therefore, the space in which memory 223 is mounted can be readily assured, and the adapter 221 is not extremely enlarged even though memory 223 is mounted.

Additionally, since the surgical instrument 240 in which key pattern portion 2411 having a specific shape is formed alone can be attached to the adapter 220, the type of surgical instrument 240 does not have to be identified in the adapter 220. Therefore, storing the control parameters of each surgical instrument 240 associated with the adapter 220 alone in memory 223 can suffice. Accordingly, a capacity of memory 223 can be reduced. Further, since the type of surgical instrument 240 does not have to be identified in the adapter 220, an electric contact and others do not have to be provided between the surgical instrument 240 and the adapter 220.

Furthermore, inconveniences such as occurrence of an erroneous operation can be avoided by restricting the surgical instruments 240 that can be attached to the adapter 220 even if the surgical instrument 240 having no control program associated with the slave control unit 400 is attached.

A configuration that types of the surgical instruments 240 can be identified in the adapter 220 may be adopted. For example, when key pattern portions 2213 having a plurality of shapes are provided to the adapter main body 221, types of the plurality of surgical instruments 240 can be mechanically determined. In this case, an identification signal associated with a shape of key pattern portion 2213 is configured to be output to the slave control unit 400 when key pattern portion 2213 comes into contact with key pattern portion 2411. Moreover, the control parameters associated with the plurality of respective types of the surgical instruments 240 are stored in memory 223. When such a structure is adopted, the slave control unit 400 can be configured in such a manner that the type of surgical instrument 240 can be determined in accordance with the identification signal from key pattern portion 2213 and the control parameters associated with the determined type of the surgical instrument 240 are read from memory 223 to control the arm 200.

Additionally, an electric contact may be provided between the surgical instrument 240 and the adapter 220 to enable electrically determining the type of surgical instrument 240. In this case, the electric contact may be possibly exposed to a sterilization gas at the time of the sterilization processing. Therefore, it is desirable to take a measure against the sterilization processing, e.g., using a metal material that can resist the sterilization processing to constitute the electrical contact.

In the configuration depicted in FIG. 6, a position in the adapter 220 at which memory 223 is arranged is not restricted in particular. However, the adapter 220 is exposed to the sterilization gas. Therefore, it is desirable to arrange memory 223 in a region that is not exposed to the sterilization gas in the adapter main body 221, bury memory 223 in the adapter main body 221, or coat memory 223 in advance, for example.

Further, in the configuration depicted in FIG. 6, the number of wiring lines is increased beyond the number of wiring lines in the configuration depicted in FIG. 3 since memory 223 is provided. On the other hand, for example, wiring line 206, wiring line 205a, and wiring line 205b are bundled by, e.g., a band 207 and connected to the slave control unit 400 in this bundled state, an influence of, e.g., enlargement of a wiring area due to an increase in the number of wiring lines can be reduced.

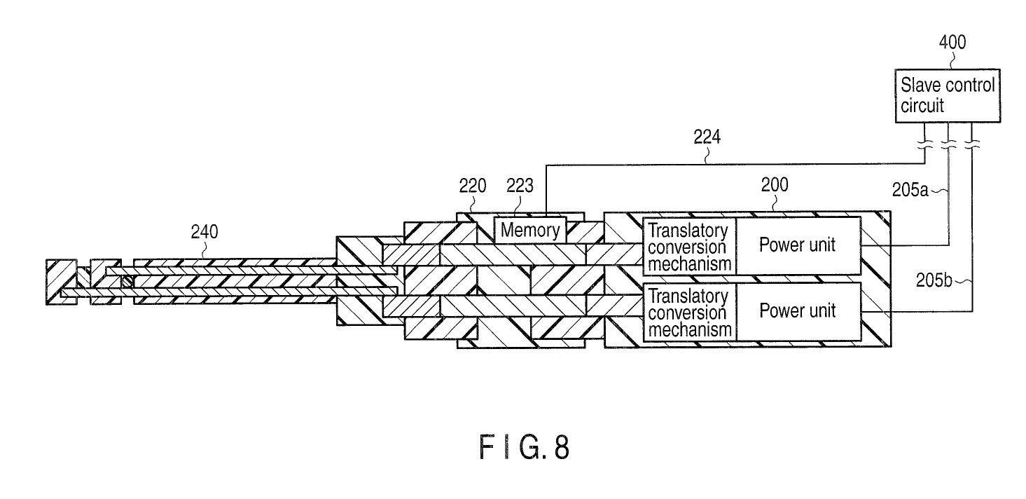

A modification of the second embodiment will now be described. FIG. 8 is a view showing a configuration of an adapter according to a modification of the second embodiment according to the invention. The example shown in FIG. 6 has a configuration that wiring line 224 connected to memory 223 is let out from the arm 200. On the other hand, in FIG. 8, a wiring line 224 connected to a memory 223 is let out from an adapter main body 221 of an adapter 220. Adopting the configuration shown in FIG. 8 enables reducing the number of wiring lines of the arm 200. Furthermore, a sufficient distance between wiring line 224 and a wiring line 205a or a wiring line 205b can be assured. Therefore, a possibility that noise is mixed in wiring line 224 when a signal is transmitted through wiring line 205a or wiring line 205b can be reduced.

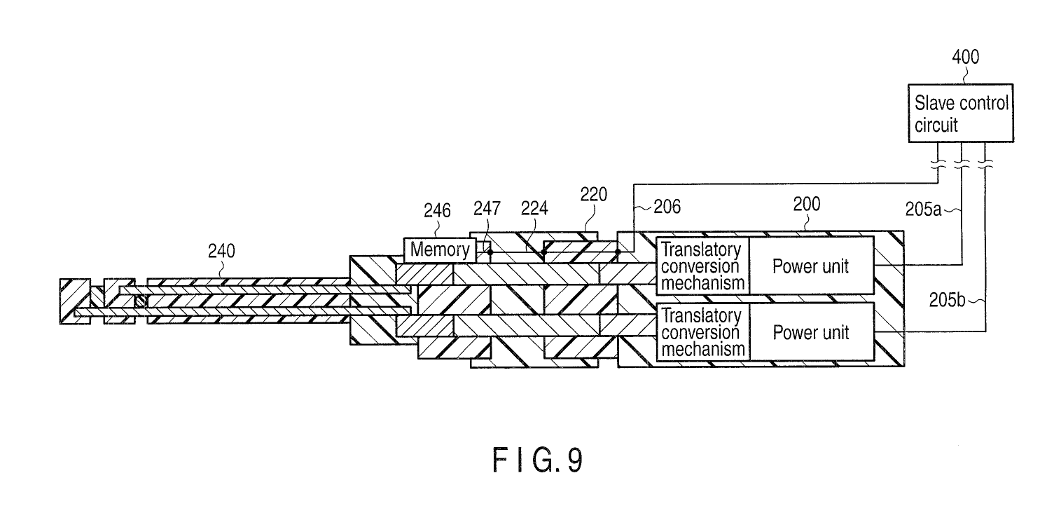

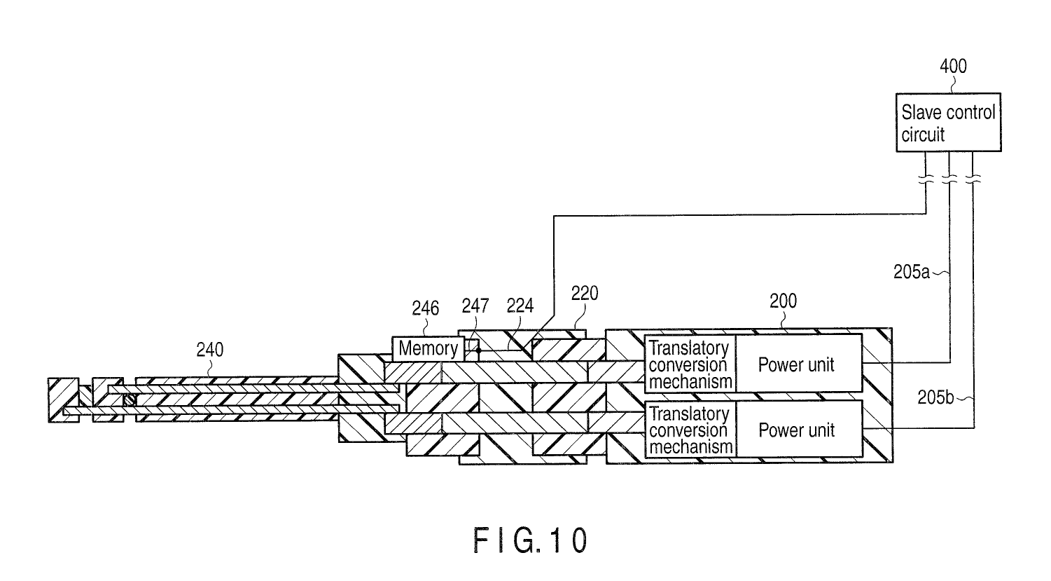

FIG. 6 and FIG. 8 show the examples where the memory is provided in the adapter 220. On the other hand, FIG. 9 and FIG. 10 show examples where a memory 246 is provided in a surgical instrument 240. FIG. 9 is associated with FIG. 6 and shows an example that a wiring line of memory 246 is let out from an arm 200. As shown in FIG. 9, memory 246 is provided to the surgical instrument 240. Memory 246 stores intrinsic information of each surgical instrument 240, e.g., calibrating information required to correct a deviation of a driving amount due to, e.g., a production tolerance at the time of producing the surgical instrument 240, or an operating time, the number of times of use, and others of the surgical instrument 240 in advance. Like memory 223, it is desirable to arrange memory 246 at a region that is not exposed to a sterilization gas in a surgical instrument main body 241.

A wiring line 247 of memory 246 is configured to be connected with wiring line 224 provided to the adapter 22n when the surgical instrument 240 is connected to the adapter 220. Wiring line 224 is configured to be connected with a wiring line 206 provided to the arm 200 when the adapter 220 is connected to the arm 200. Wiring line 206 is let out from an arm main body 201 and connected to a slave control unit 400 together with wiring lines 205a and 205b required to drive power units 202a and 202b.

When memory 246 is provided to the surgical instrument 240 as shown in FIG. 9, intrinsic information of each surgical instrument can be stored in memory 246 to be used. As a result, optimum driving control associated with a specification and others of each surgical instrument 240 can be carried out in the slave control unit 400. Furthermore, when operating times or the numbers of times of use of the surgical instruments 240 are stored in memory 246, management of a maintenance time and others of each surgical instrument 240 can be carried out.

FIG. 10 is associated with FIG. 8 and shows an example of pulling the wiring line of memory 246 from the adapter 220. As shown in FIG. 10, wiring line 247 of memory 246 is configured to be connected to wiring line 224 provided to the adapter 220 when the surgical instrument 240 is connected to the adapter 220. Wiring line 224 is let out from the adapter main body 221 of the adapter 220 and connected to the slave control unit 400. When such a configuration as depicted in FIG. 10 is adopted, intrinsic information of each surgical instrument can be stored in memory 246 and used, and the number of wiring lines of the arm 200 can be reduced. Moreover, a sufficient distance between wiring line 224 and wiring line 205a or wiring line 205b can be maintained, thus reducing a possibility that noise is mixed in wiring line 224 when a signal is transmitted through wiring line 205a or wiring line 205b.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A surgical power transmission adapter that is interposed between a surgical instrument and a power unit configured to drive the surgical instrument and connects the surgical instrument to the power unit, comprising:
a power transmission unit that includes a first region which is in contact with a clean region that is subjected to sterilization processing and a second region which is in contact with an unclean region that is not subjected to the sterilization processing, and that is configured to make translatory movement to transmit power generated in the power unit to the surgical instrument,
wherein a range of the translatory movement of the power transmission unit is set in such a manner that the first region is placed in the clean region and the second region is placed in the unclean region even if the power transmission unit makes the translatory movement.

2. The adapter according to claim 1, wherein the first region is a region that is in contact with the surgical instrument, and the second region is a region that is in contact with the power unit.

3. The adapter according to claim 1, further comprising an adapter main body which accommodates the power transmission unit to have a sterilization space where the sterilization processing is performed.

4. The adapter according to claim 1, further comprising a surgical instrument identification unit configured to identify the surgical instrument.

5. The adapter according to claim 4, wherein the surgical instrument identification unit comprises a memory storing information required to identify the surgical instrument.

6. The adapter according to claim 5, wherein a wiring line of the memory and a wiring line of the power unit are connected to a control circuit for the memory and the power unit in a bundled state.

7. The adapter according to claim 5, wherein a wiring line of the memory and a wiring line of the power unit are connected to a control circuit for the memory and the power unit in a separated state.

8. The adapter according to claim 4, wherein the surgical instrument identification unit comprises an attachment portion formed in the surgical power transmission adapter to enable attachment of a specific type of surgical instrument alone to the surgical power transmission adapter.

9. The adapter according to claim 4, wherein the surgical instrument identification unit comprises: a memory storing information required to identify the surgical instrument; and an attachment portion formed in the surgical power transmission adapter to enable attachment of a specific type of surgical instrument alone to the surgical power transmission adapter.

10. A medical manipulator system comprising:
a surgical power transmission adapter comprising:
a power transmission unit that includes a first region which is in contact with a clean region that is subjected to sterilization processing and a second region which is in contact with an unclean region that is not subjected to the sterilization processing, and that is configured to make translatory movement,
a range of the translatory movement being set in such a manner that the first region is placed in the clean region and the second region is placed in the unclean region even if the power transmission unit makes the translatory movement;
a power unit that is connected to the surgical power transmission adapter and configured to transmit generated power by the translatory movement of the power transmission unit;
a surgical instrument that is connected to the surgical power transmission adapter and configured to be driven by the power transmitted based on the translatory movement of the power transmission unit; and
a control circuit that is connected to the power unit and configured to control operations of the surgical instrument by controlling the power unit.

11. The system according to claim 10, wherein the first region is a region that is in contact with the surgical instrument, and the second region is a region that is in contact with the power unit.

12. The system according to claim 10, wherein the surgical power transmission adapter further comprises an adapter main body which accommodates the power transmission unit to have a sterilization space where the sterilization processing is performed.

13. The system according to claim 10, wherein the surgical power transmission adapter further comprises a surgical instrument identification unit configured to identify the surgical instrument.

14. The system according to claim 13, wherein the surgical instrument identification unit comprises a memory storing information required to identify the surgical instrument.

15. The system according to claim 14, wherein a wiring line of the memory and a wiring line of the power unit are connected to a control circuit for the memory and the power unit in a bundled state.

16. The system according to claim 14, wherein a wiring line of the memory and a wiring line of the power unit are connected to a control circuit for the memory and the power unit in a separated state.

17. The system according to claim 13, wherein the surgical instrument identification unit comprises an attachment portion formed in the surgical power transmission adapter to enable attachment of a specific type of surgical instrument alone to the surgical power transmission adapter.

18. The system according to claim 13, wherein the surgical instrument identification unit comprises: a memory storing information required to identify the surgical instrument; and an attachment portion formed in the surgical power transmission adapter to enable attachment of a specific type of surgical instrument alone to the surgical power transmission adapter.
